(19) United States
(12) Patent Application Publication
(10) Pub. No.: US 2024/0052166 A1
(43) Pub. Date: Feb. 15, 2024

(54) Method and Device for Producing Hollow Spheres Composed of Collagen and Collagen Derivatives and Hollow Spheres Composed of Collagen and Collagen Derivatives

(71) Applicant: FILK Freiberg Institute gGmbH, Freiberg (DE)

(72) Inventors: Ina Prade, Hainichen (DE); Michael Meyer, Dresden (DE); Claudia Krumbiegel, Dittmannsdorf (DE)

(21) Appl. No.: 18/232,840

(22) Filed: Aug. 11, 2023

(30) Foreign Application Priority Data
Aug. 15, 2022 (DE) 102022120500.2

(51) Int. Cl. C08L 89/00

(52) U.S. Cl. CPC C08L 89/00

(57) ABSTRACT

Hollow spheres are produced having a wall composed of collagen and at least one collagen derivative, wherein the cavity is filled with at least one gas. For this purpose, at least one suspension or solution of collagen and at least one collagen derivative, at least one separation liquid and at least one crosslinking liquid are arranged in a container, followed by introduction of a gas mixture via a gas supply device, as a result of which gas-filled hollow spheres having a collagen/collagen derivative wall are formed and are supplied to the separation liquid and then to the crosslinking liquid.

### CROSS REFERENCE TO RELATED APPLICATIONS

This application claims the priority of DE 102022120500.2 filed on 2022 Aug. 15; this application is incorporated by reference herein in its entirety.

### BACKGROUND

The present invention relates to the fields of medical and laboratory technology and of cell biology and relates to a method and device for producing hollow spheres composed of collagen and collagen derivatives and to hollow spheres composed of collagen and at least one collagen derivative that are produced in such a manner. The hollow spheres composed of collagen and at least one collagen derivative that are produced by means of the method and device according to the invention can be used, for example, as in vitro models, or for tissue engineering, in the field of pneumology, as a drug delivery and cell carrier system in the context of medical therapies, as an adhesion and compartmentalization system for high-density cultures in the production of biologicals and as a capsule for biosensor elements.

While in vivo experiments on animal models have been the focus of these studies in past decades, doubts are now being cast on the benefit of these regulatorily prescribed tests. The use of alternative models is supported by the fact that many physiological and pathological processes in humans differ significantly from animals. Furthermore, ethical issues in connection with animal experiments are also increasingly being discussed, and searches are being made for substitute methods.

Using in vitro models, it is possible to model complex phenomena of the human body under simplified, easily controllable and readily available conditions. They make it possible to assess the efficacy and safety of medicaments and are thus an important tool in developing new therapies. Besides predictability of the effect, human tissue models can be used supportively for clarifying mechanisms of the effect of drug substances or disease causes. Nevertheless, it is known that this simplification also entails constraints. Not only the cell types used, but also especially design and complexity influence the physiological relevance of the model.

In order to achieve a high degree of similarity of an in vitro model to, for example, the alveoli of the human body, various components of the lung are physiologically relevant. Moreover, when designing an in vitro model, an elastic substrate composed of ECM components for the adhesion of cells should be taken into account, as should the possibility of mechanical stimulation that leads to movement of the alveolar barrier during breathing.

Generation of a realistic in vitro model requires that as many characteristic features of a tissue as possible be simulated. In the past, the generation of a round geometry composed of collagen has been investigated especially for use in drug delivery. There are, however, also numerous tissues in the human body that have a round shape, for example the alveoli, the bladder, or the capsular bag of the eye.

Various approaches for generating and producing in vitro models using collagen are already known from the prior art and describe the production of either completely filled collagen spheres, so-called microspheres, or hollow spheres.

WO 2021 141 595 A1 discloses a method for producing microcarriers composed of collagen for use in wound healing. The microcarriers consist of collagen, a stabilizing fatty acid such as stearic acid, and a liquid core which can be enriched with oxygen and a pro-healing active ingredient. Additional stabilization is achieved by the addition of phospholipids, mono- or polysaccharides, polyols, glycoproteins and glycolipids, or phosphoproteins.

The microcarriers are produced by initially charging an aqueous solution of the aforementioned components in a container, followed by introduction of gas (e.g. oxygen) via the liquid phase. Bubbles are formed at the gas-liquid boundary as a result of the application of pressure and stimulation, for example by means of ultrasound or stirring. The spheres are stabilized in the aqueous phase as a result of the cooling of the container. This then yields a collection of spheres of different sizes (between 0.5 and 30 μm).

WO 2014 025 312 discloses the production of gelatin microparticles filled with cells. To this end, the cells are added to a gelatin solution and mixed with an oil. The emulsion is stirred at low temperatures, forming small particles filled with cells. The particles are separated from the oil and are either used singly or coated with alginate. After gelling of the alginate, the gelatin is dissolved away by heat and the cells remain in an alginate scaffold having round cavities.

EP 1 707 260 A1 describes a method for producing crosslinked microspheres composed of collagen. To this end, an acidic collagen solution is admixed with an emulsifier such as polyvinyl alcohol and combined with a hydrophobic liquid (chloromethane or ethyl acetate) under conditions which lead to the formation of a water-in-oil emulsion. The water-in-oil emulsion is added to a buffered alcoholic solution. This yields a dispersion having a solid phase composed of reconstituted fibrillated collagen. Removal of the liquid phase yields collagen microspheres, which are freeze-dried and subsequently chemically crosslinked.

WO 2019 136 453 A1 discloses the generation of a 3D cell culture model for simulation of the alveoli of the lungs. This involves producing microspheres based on various chemical components such as PEG, cell adhesion-promoting peptide sequences and crosslinkers and admixing them with cells with the aid of a microfluidic device. The spheres are additionally loaded with magnetic particles in order to initiate aggregation of multiple spheres under cell culture conditions. The spheres consist of a capsule and an interior. The latter can be digested by enzymes of the cells. This yields cell-loaded hollow spheres. The spheres are incorporated in a matrix, the mechanical strength of which can be varied. To this end, photosensitive hydrogels such as methacrylated gelatin are used.

Further solutions are known from the prior art for forming 3D lung models.

WO 2018 122 219 A1 and WO 2009 048 661 A1 disclose cell culture models in planar format, in which the cells grow as multiple layers on a porous membrane.

Furthermore, EP 2450707B1 discloses the culturing of various cells classified under lung tissue, without said cells having an artificial scaffold matrix.

It is additionally also known from WO 2014 018 691 A1 to generate 3D spheres composed of lung progenitor and epithelial cells. The growth factors and signalling substances with which the cells must be cultured in non-adhesive culture vessels in order to automatically form spheres or hollow spheres are described.

The cells in the body are in a complex environment. Not only are extracellular matrix proteins to be found, but also mechanical forces which act on the cells and influence the behaviour and response of the cells. In vitro mechanical stimulation has hitherto not been realized on 3D tissues containing lumens.

One disadvantage in the prior art is that the in vitro modelling of hollow spheres and the mechanical simulation thereof is unsatisfactory. Another disadvantage is that the proposed methods are associated with high costs and the use of complicated laboratory technology with unsatisfactory reproducibility of the models and a wide size distribution of the hollow spheres, which, furthermore, are not present singly, but as an agglomerate or as a foam and thus as an accumulation of hollow spheres of different sizes.

Another disadvantage with the prior in vitro models is that use is made of, for example, emulsifiers, surfactants, stabilizers or the like that can lead to undefinable and undesired interactions with cells.

### SUMMARY

The invention relates to the fields of medical and laboratory technology and of cell biology and relates to a method and device for producing hollow spheres composed of collagen and collagen derivatives and to hollow spheres produced in such a manner. It is an object of the invention to provide a cost-effective and reproducible device and method for producing hollow spheres as in vitro models and to allow realistic simulation of human cells and alveoli in vitro.

The invention provides a method and device for producing hollow spheres having a wall composed of collagen and at least one collagen derivative, wherein the cavity is filled with at least one gas. For this purpose, at least one suspension or solution of collagen and at least one collagen derivative, at least one separation liquid and at least one crosslinking liquid are arranged in a container, followed by introduction of a gas mixture via a gas supply device, as a result of which gas-filled hollow spheres having a collagen/collagen derivative wall are formed and are supplied to the separation liquid and then to the crosslinking liquid.

### DETAILED DESCRIPTION

It is an object of the present invention to provide a cost-effective, simple, rapid and reproducible device and method for producing singly handleable hollow spheres as in vitro models. It is a further object of the invention to provide hollow spheres as in vitro models which allow improved realistic simulation of human cells and alveoli in vitro.

The object is solved by the technical features of the main claim(s). Advantageous embodiments are subject matter of the dependent claims, the invention also including combinations of the individual dependent claims in the sense of an “and” link so long as they are not mutually exclusive.

The object of the invention is solved by a method for producing hollow spheres composed of collagen and collagen derivatives, in which at least one suspension or solution of collagen and at least one collagen derivative, at least one separation liquid of higher viscosity and lower density compared to the suspension and at least one crosslinking liquid of lower viscosity and lower density compared to the separation liquid are arranged in a container, wherein the separation liquid is arranged as a phase-separation layer between the suspension and the crosslinking liquid, followed by introduction of a gas mixture into the suspension or solution via at least one gas supply device, as a result of which hollow spheres filled with this gas and having a collagen/collagen derivative wall are formed from the introduced gas bubbles and are supplied to the separation liquid, followed by penetration of the separation liquid by the hollow spheres and followed by supply of the hollow spheres to the crosslinking liquid, wherein the hollow spheres are stabilized and singularized in the crosslinking liquid, and followed by removal of the hollow spheres formed from the crosslinking liquid, wherein further treatments of the hollow spheres can be carried out afterwards.

Advantageously, the container is temperature-controlled at least in the region of the suspension or solution via an additional thermal source and/or by addition of at least one additive, wherein, particularly advantageously, urea, guanidine hydrochloride and/or other hydrotropically acting additives are added as the additive.

Particularly advantageously, water-soluble oligomers or polymers, preferably carbohydrates and/or polysaccharides, are added for regulation of density and viscosity.

In an advantageous embodiment of the method, a controllable gas supply device is used, by means of which the suppliable gas volume of the gas mixture is adjustable and the outer diameter of the hollow spheres composed of collagen and collagen derivatives is variable as a result.

In addition, it is advantageous if the suspension or solution is used at a proportion of 1% by weight to 50% by weight of collagen and collagen derivative.

Furthermore, it is advantageous if a non-water-miscible liquid is used as the separation liquid.

In an advantageous embodiment of the method, the separation liquid is at least partially removed from the hollow sphere produced by at least one cleaning operation with a solvent, wherein, particularly advantageously, an alcoholic solution, particularly advantageously ethanol, is used as the solvent.

Moreover, it is advantageous if at least one cell suspension, tissue cells and/or microorganisms is incubated and/or injected in the hollow sphere produced and/or on its wall on the inside and/or outside.

Also advantageously, the hollow spheres, following removal from the crosslinking liquid, are supplied to a post-crosslinking liquid for post-crosslinking, wherein it is particularly advantageous if crosslinking enzymes, preferably transglutaminase, and/or formaldehyde, bifunctional aldehydes, carbodiimides and/or isocyanates in aqueous solution are used in the post-crosslinking liquid.

It is also advantageous if the collagen derivative(s) is/are modified by acrylates, methacrylates and/or vinyl compounds.

It is additionally also advantageous if the crosslinking and/or the post-crosslinking is effected physically by electromagnetic or particle radiation.

The object of the invention is also solved by a device for carrying out the above-described method, comprising a container having at least one gas supply device, wherein a suspension or solution composed of collagen and at least one collagen derivative, a separation liquid of higher viscosity and lower density compared to the suspension and a crosslinking liquid of lower viscosity and lower density compared to the separation liquid are present in the container, wherein the gas supply device is realized in the region of the arranged suspension, and wherein a separation device is arranged in the region of the crosslinking liquid.

Advantageously, a thermal source is arranged inside and/or outside the container at least in the region of the suspension or solution.

It is likewise advantageous if the at least one gas supply device is provided in the region of the container base or in the container lateral wall.

Also advantageously, two or more gas supply devices which allow gas supply into the suspension at different container heights are present.

Moreover, it is advantageous if the separation device is a funnel-shaped component, overflow arrangement, skimming device, suction device and/or a pneumatic device.

Moreover, the object of the invention is solved by a hollow sphere having a multiaxially and elastically deformable wall composed of collagen and at least one collagen derivative, wherein at least the cavity formed is filled with at least one gas.

It is advantageous if the collagen contains one or more different collagen types.

Advantageously, present in the hollow sphere is fibrillated collagen and gelatin as the collagen derivative.

In an advantageous embodiment of the hollow sphere, at least the cavity contains a liquid, microorganisms, solids and/or cellular tissue.

Furthermore, it is advantageous if the hollow sphere has an outer diameter of 0.2 mm to ≤1.5 cm.

Additionally also advantageously, the wall of the hollow sphere has a layer thickness of 5 μm to ≤500 μm, preferably 10 μm to 200 μm.

The present invention provides a method and device for producing hollow spheres composed of collagen and at least one collagen derivative as an in vitro model that is cost-effective, simple, rapid and reliable and allows high reproducibility. Additionally provided are hollow spheres as in vitro models that exhibit improved realistic simulation and mechanical stimulation of human cells and alveoli with high similarity to the human alveolus with matched mechanical and physical properties.

In the following description, the exemplary embodiment and the claims, the facts essential to the invention are defined as follows:

In the context of the invention, collagen is understood to mean the family of structural proteins preferably to be found in the connective tissue of higher animals, characterized by the repeating amino acid sequence (triplet) glycine-X-Y, wherein X can often be proline and Y can often be hydroxyproline. In this family of structural proteins, the individual members, which differ little in their sequence within a species, are represented by Roman numerals (I . . . XXVIII), the important structural proteins in terms of quantity belonging to collagens I to V. The regular repetition of the glycine at every third position gives rise to a structure which is unique to collagen, but which need not be distributed over the entire chain length. The primary structure of the polypeptide chains forms a left-handed helix.

Three of these polypeptide chains can associate in regions of the triplets to form a right-handed triple helix. Collagen molecules consist of triple-helically associated collagen peptide chains. The collagen molecules can be present as a solution.

In the context of the invention, fibrillar collagen is understood to mean collagen triple helices which have associated to form higher structures having a defined repeating so-called transverse striation of 63-70 nm. It is depictable under an atomic force microscope, a scanning electron microscope or else a transmission electron microscope.

In the context of the invention, collagen derivatives are understood to mean chemical and structural derivatives of the above-described collagen which are preferably obtained by technical processes from animal tissue or from collagen formed by genetic modification from microorganisms or else from chemically synthesized peptides and in which the triple-helical structure is not present or only partially present.

In the context of the invention, a suspension is understood to mean a soluble preparation of collagen or collagen derivatives that is not completely soluble in solvent.

In the context of the invention, a solution is understood to mean a preparation of collagen or collagen derivatives in solvent, preferably water, organic acids or aqueous solvents admixed with additives, which preparation is either completely filterable or the filtrate of a preparation.

In the context of the invention, a separation liquid is understood to mean a liquid immiscible with water or aqueous solvents, for examples oils.

In the context of the invention, a crosslinking liquid is understood to mean a liquid which contains components which crosslink collagen or collagen derivatives, for example chemical crosslinkers or else enzymes.

According to the invention, the hollow spheres composed of collagen and at least one collagen derivative are produced by providing, in a first method step, a container in which a suspension or solution containing collagen and at least one collagen derivative, at least one separation liquid of higher viscosity and lower density compared to the suspension and at least one crosslinking liquid of lower viscosity and lower density compared to the separation liquid are arranged.

According to the invention, mixing of the suspension and the crosslinking liquid within the container is avoided by arranging the separation liquid as a phase-separation layer between the suspension and the crosslinking liquid in order to avoid mixing of the suspension and the crosslinking liquid.

According to the invention, uniform and reproducible individual hollow spheres composed of collagen and at least one collagen derivative are formed by introduction of a gas into the suspension or solution via at least one gas supply device, as a result of which gas-filled hollow spheres having a collagen/collagen derivative wall are formed from the gas bubbles introduced into the suspension and are then supplied to the separation liquid.

In an advantageous embodiment of the method, it is conceivable that a controllable gas supply device is used, by means of which the suppliable gas volume of the gas mixture is adjustable and the size and the outer diameter of the hollow spheres can be substantially influenced as a result. It is thus possible to obtain in a controlled manner hollow spheres composed of collagen and at least one collagen derivative having outer diameters which are freely variable, but reproducible in a defined manner.

It is also conceivable that multiple controllable gas supply devices are used. Firstly, by using multiple gas supply devices, the yield of hollow spheres composed of collagen and collagen derivative per unit time can be increased. Secondly, there is the possibility of providing the gas supply devices at varying heights of the container. Thus, it is conceivable that a gas mixture is supplied to the suspension or solution via a first gas supply device at the base of the container, whereas a second gas supply device in the lateral container wall realizes a further gas supply into the suspension or solution. Hollow spheres of different wall thicknesses can be formed owing to the different rise heights, thereby allowing high flexibility of the method and device with respect to the size of the hollow spheres.

According to the invention, the hollow spheres formed in the suspension or solution penetrate the separation liquid and are then supplied to a crosslinking liquid, wherein the hollow spheres are stabilized in the crosslinking liquid, and the hollow spheres formed are then removed from the crosslinking liquid singly or as agglomerates, wherein further treatments of the hollow spheres, for example post-crosslinking or colonization by or incubation with cells, can be carried out afterwards.

It has been found that the viscosity of especially the suspension or solution is an important parameter for the specific formation of the hollow spheres composed of collagen and at least one collagen derivative. Thus, it has been found to be advantageous if the viscosity of the suspension is set by specific temperature control in a predetermined temperature range.

Advantageously, it is proposed that at least the suspension is temperature-controlled via an additional thermal source and/or by addition of at least one additive, for example urea, thereby, firstly, setting the required viscosity of the suspension, in particular the collagen derivative, and, secondly, preventing denaturation of the triple-helical collagen. To reach the desired viscosity, the suspension or solution is advantageously adjusted to a temperature of 25° C. to 40° C.

As a result, a defined buoyancy velocity of the gas bubbles within the suspension or solution can be set and the formation of the wall thicknesses of the hollow spheres of advantageously 5 μm to 500 μm, preferably 10 μm to 200 μm, can thus be influenced in a controlled manner.

Such a thermal source can be provided, for example, within the container in the region of the introduced suspension However, it is also possible that the container is heated from the outside, for example by introduction of the container into a temperature-controlled water bath or by transfer of heat from an external thermal radiation source by means of electromagnetic waves.

To form particularly elastic and mechanically stressable hollow spheres composed of collagen and collagen derivative, it has been found to be advantageous if the suspension or solution used comprises a proportion of 1% by weight to 50% by weight of collagen and collagen derivative, the collagen derivative used being particularly advantageously denatured collagen triple helices, for example gelatin or collagen hydrolysate.

To implement reliable phase separation and to prevent mixing of suspension or solution and crosslinking liquid, it is proposed that a separation liquid is arranged in the container between the suspension or solution and the crosslinking liquid, which separation liquid is advantageously a non-water-miscible liquid and has a different density in relation to both the suspension or suspension and the crosslinking liquid.

Advantageously, in a downstream method step, the crosslinking liquid can be at least partially removed from the hollow sphere produced by at least one cleaning operation with a solvent. The at least partial removal of the crosslinking liquid from the surface of the hollow spheres offers the advantage that an improved substrate surface is provided, on which, for example, tissue cells or microorganisms can be established. Advantageously, the cleaning solution used in this connection is an alcoholic solution, particularly advantageous examples being ethanol or isopropyl alcohol.

In a further advantageous embodiment of the method, the hollow spheres or agglomerates, following removal from the crosslinking liquid, can be supplied to a post-crosslinking liquid for an additional post-crosslinking step in order to achieve a further improvement in the multiaxial compression properties.

Advantageously, the post-crosslinking liquid can be used with crosslinking enzymes, preferably transglutaminase, and/or formaldehyde, bifunctional aldehydes, carbodiimides and/or isocyanates in aqueous solution.

The hollow spheres produced from collagen and at least one collagen derivative offer the possibility that, for example, a cell suspension, tissue cells and/or microorganisms and/or a liquid containing bioactive substances can be injected into the interior of the hollow spheres or, for example, tissue cells and/or microorganisms can be incubated on the wall of the hollow spheres on the inside and outside without damaging or destroying the wall of the hollow sphere, since the injection hole recloses by itself owing to the elastic properties.

According to the invention, a device for carrying out the above-described method is also provided. The device comprises a container having at least one gas supply device, wherein a suspension or solution composed of at least collagen and at least one collagen derivative, a separation liquid of higher viscosity and lower density compared to the suspension and a crosslinking liquid of lower viscosity and lower density compared to the separation liquid are present in the container. The separation liquid is to be understood as a phase-separation liquid which spatially separates the suspension or solution and the crosslinking liquid.

According to the invention, the gas supply device is realized in the region of the arranged suspension or solution.

Moreover, in the region of the crosslinking liquid, the device comprises a separation device, by means of which the hollow spheres or agglomerates produced can be withdrawn from the container. Advantageously, such a separation device can be a funnel-shaped component, overflow arrangement, skimming device, suction device and/or a pneumatic device.

In an advantageous embodiment of the device, a defined viscosity of the suspension or solution can be set by arranging a thermal source inside and/or outside the container at least in the region of the suspension or solution.

The supply of the gas mixture into the suspension composed of collagen and at least one collagen derivative can be effected, for example, via an opening in the base of the container or in the lateral wall of the container, which especially influences the possible rise height of the gas bubbles or hollow spheres within the suspension or solution and thus the formation of the wall thickness of the hollow spheres.

In an advantageous embodiment of the device, two or more gas supply devices which realize gas supply into the suspension at different container heights can be present. As a result, two or more different hollow spheres of different wall thicknesses are produced in one method step, thereby improving the variability and efficiency of the device and method.

Preferably, the device according to the invention can provide hollow spheres composed of collagen and at least one collagen derivative having an outer diameter of 0.2 mm to 1.5 cm, wherein the layer thickness of the wall can be 5 μm to 500 μm, preferably 10 μm to 200 μm.

The method according to the invention and the device according to the invention provide novel hollow spheres having a multiaxially and elastically deformable wall composed of collagen and at least one collagen derivative, wherein the cavity formed is filled with at least one gas.

The novel hollow spheres have the main advantage that the wall of the hollow sphere is stable as a result of the combination of collagen and at least one collagen derivative, thereby preventing collapse or bursting of the hollow sphere.

Collagen is the most common protein in the human body, and it is biocompatible and thus better suited to in vitro models than other, synthetic polymers. The collagen structure is crucial for the cell and has particularly dimensionally stable properties. Moreover, cells are capable of directly adhering to the collagen by means of cell adhesion molecules, and so the collagen used in hollow spheres forms the appropriate substrate for in vitro models. Besides its function as a structural molecule, collagen exercises important functions in the control of cell growth. Advantageously, fibrillated collagen can be used, which has improved stability properties compared to triple-helical collagen, forms the main constituent of the extracellular matrix in all multicellular organisms and is thus of great importance for biomedical purposes.

Besides collagen, which can be triple-helical and/or, advantageously, fibrillar, at least one collagen derivative is also used according to the invention as cell substrate. It can be generated by heating collagen and is thus a degradation variant of the extracellular matrix component. In a preferred embodiment of the invention, gelatin is used as the collagen derivative. In contrast to the triple-helical collagen, gelatin has a foam-stabilizing effect, but has nevertheless the typical amino acid sequences for the recognition and adherence of cells.

Advantageously, the triple-helical collagen used can be type I, type II and/or type III collagen or else other types in combination with at least one collagen derivative in an aqueous suspension or solution. The components are very readily miscible and moreover biocompatible.

The wall of the hollow spheres composed of collagen and at least one collagen derivative that are formed, on which cells are colonized, ultimately consists of two adjacent cell layers separated by an adhesion matrix based on the biomaterial collagen and its derivatives. The thin elastic wall allows the exchange of oxygen, nutrients and waste products and allows multiaxial compression in accordance with the mechanical and physiological properties of, for example, human alveoli.

The technical advantages and effects of the invention are that
a very simple and cost-effective method and device for producing hollow spheres composed of collagen and collagen derivatives is provided,
an ideal in vitro model similar to, for example, the human alveoli is provided,
the hollow spheres composed of collagen and collagen derivatives are stable with respect to shear stress, cannulation, culture media and cells,
the hollow spheres composed of collagen and collagen derivatives are biocompatible and are colonizable by different cells on the inside and on the outside,
their gas space is reachable by means of injection,
the parameters of the physiological properties of the hollow spheres can be influenced by simple means,
the hollow spheres generated can be used in a wide range of technical and medical applications,
in vitro mechanical stimulation on 3D tissues having lumens is made possible, and
it is possible for the first time to generate hollow collagen/collagen derivative spheres in a simple process and without the aid of synthetic polymers.

The invention will be more particularly elucidated below on the basis of an exemplary embodiment.

### DETAILED DESCRIPTION OF THE PREFERRED EMBODIMENTS

To produce individual hollow spheres composed of collagen and collagen derivatives from a water-based suspension containing collagen and type A gelatin having a Bloom value of 300, 0.04% by weight of collagen dissolved under acidic conditions (2 mg/ml collagen in 0.01 M HCl), 1% by weight of collagen fibre suspension and 26.7% by weight of aqueous gelatin solution heated to 40° C. are mixed and introduced into a transparent tube of approx. 35 cm in length to give a liquid column of 8 cm in height. The tube is heated via an additional thermal radiation source, such that the suspension is adjusted to a temperature of 40° C. A layer of castor oil of 5 cm in height is applied as separation liquid to the collagen/gelatin suspension of approx. 5 cm in height. This separates the crosslinking liquid arranged on top, which crosslinking liquid is an oil phase of lower density and low viscosity (paraffin oil containing transglutaminase, 1 g of transglut. in 10 ml of oil), from the collagen/gelatin suspension.

At the bottom end of the tube, a sterile oxygen-containing gas mixture in the form of air is introduced via a flexible tube. The collagen mixture is adjusted to a temperature of 40° C. in a water bath in order to keep the gelatin liquid and low in viscosity.

To form the hollow spheres from the mixture of collagen and gelatin, a syringe is used to conduct a defined air volume into the flexible tube and, from there, into the suspension. The air bubbles that are introduced and formed rise within the suspension and are coated with the mixture of collagen and gelatin at the same time. Thereafter, they enter through the separation liquid into the crosslinking liquid, in which the wall of the hollow sphere is stabilized. After the generated hollow spheres composed of collagen and collagen derivatives have passed through the crosslinking liquid, they enter a fitted funnel, from which the resulting hollow spheres are withdrawn. Hollow spheres composed of collagen and gelatin and having a uniform outer diameter of 300 μm are now available, which hollow spheres have substantially identical outer diameters owing to the continuous and constant introduced air volume. From the removed hollow spheres, the excess oil of the separation liquid and/or crosslinking liquid is removed from the surface of the outer wall of the hollow spheres composed of collagen and collagen derivatives by placement in ethanol. The ethanol contains a crosslinking substance which further stabilizes the wall of the hollow sphere. After repeated washing with culture medium, the hollow spheres are loaded with human cells in order to form the alveolar barrier in the form of a lung epithelium layer, a layer of endothelial cells and the basement membrane. For this purpose, one tissue cell type in the form of a suspension (culture medium+lung epithelial cells) is first injected into the interior of the hollow spheres by means of a cannula. The filled hollow spheres are then cultured in a CO₂ incubator (37° C., 95% air humidity) for 24 h. During this, the cells adhere to the inner wall. The hollow spheres can be colonized homogeneously if they are turned regularly at least within the first 2 to 4 hours.

This is followed by incubation of the hollow spheres composed of collagen and collagen derivatives with a cell suspension composed of primary endothelial cells, which show complete colonization of the surface. For this purpose, the hollow spheres loaded with cells are immersed into a further cell suspension containing a different cell type and cultured therein for up to 24 h (in a CO₂ incubator at 37° C. and 95% air humidity). Thereafter, the hollow spheres colonized by cells are transferred into a culture vessel containing fresh medium.

When pressure is applied cyclically to a hollow sphere floating in liquid, elastic properties similar to the human alveoli can be observed with recurring compression and relaxation of the geometry of the hollow spheres composed of collagen and collagen derivatives.

1. Method for producing hollow spheres composed of collagen and collagen derivatives, in which at least one suspension or solution of collagen and at least one collagen derivative, at least one separation liquid of higher viscosity and lower density compared to the suspension and at least one crosslinking liquid of lower viscosity and lower density compared to the separation liquid are arranged in a container, wherein the separation liquid is arranged as a phase-separation layer between the suspension and the crosslinking liquid, followed by introduction of a gas mixture into the suspension or solution via at least one gas supply device, as a result of which hollow spheres filled with this gas and having a collagen/collagen derivative wall are formed from the introduced gas bubbles and are supplied to the separation liquid, followed by penetration of the separation liquid by the hollow spheres and followed by supply of the hollow spheres to the crosslinking liquid, wherein the hollow spheres are stabilized and singularized in the crosslinking liquid, and followed by removal of the hollow spheres formed from the crosslinking liquid, wherein further treatments of the hollow spheres can be carried out afterwards.

2. Method according to claim 1, in which the container is temperature-controlled at least in the region of the suspension or solution via an additional thermal source and/or by addition of at least one additive.

3. Method according to claim 2, in which urea, guanidine hydrochloride and/or other hydrotropically acting additives are added as the additive.

4. Method according to claim 2, in which water-soluble oligomers or polymers, preferably carbohydrates and/or polysaccharides, are added for regulation of density and viscosity.

5. Method according to claim 1, in which a controllable gas supply device is used, by means of which the suppliable gas volume of the gas mixture is adjustable and the outer diameter of the hollow spheres composed of collagen and collagen derivatives is variable as a result.

6. Method according to claim 1, in which the suspension or solution is used at a proportion of 1% by weight to 50% by weight of collagen and collagen derivative.

7. Method according to claim 1, in which a non-water-miscible liquid is used as the separation liquid.

8. Method according to claim 1, in which the separation liquid is at least partially removed from the hollow sphere produced by at least one cleaning operation with a solvent.

9. Method according to claim 8, in which an alcoholic solution, particularly advantageously ethanol, is used as the solvent.

10. Method according to claim 1, in which at least one cell suspension, tissue cells and/or microorganisms is incubated and/or injected in the hollow sphere produced and/or on its wall on the inside and/or outside.

11. Method according to claim 1, in which the hollow spheres, following removal from the crosslinking liquid, are supplied to a post-crosslinking liquid for post-crosslinking.

12. Method according to claim 11, in which crosslinking enzymes, preferably transglutaminase, and/or formaldehyde, bifunctional aldehydes, carbodiimides and/or isocyanates in aqueous solution are used in the post-crosslinking liquid.

13. Method according to claim 1, in which the collagen derivative(s) is/are modified by acrylates, methacrylates and/or vinyl compounds.

14. Method according to claim 1, in which the crosslinking and/or the post-crosslinking is effected physically by electromagnetic or particle radiation.

15. Device for carrying out the method according to claim 1, comprising a container having at least one gas supply device, wherein a suspension or solution composed of collagen and at least one collagen derivative, a separation liquid of higher viscosity and lower density compared to the suspension and a crosslinking liquid of lower viscosity and lower density compared to the separation liquid are present in the container, wherein the gas supply device is realized in the region of the arranged suspension, and wherein a separation device is arranged in the region of the crosslinking liquid.

16. Device according to claim 15, in which a thermal source is arranged inside and/or outside the container at least in the region of the suspension or solution.

17. Device according to claim 15, in which the at least one gas supply device is provided in the region of the container base or in the container lateral wall.

18. Device according to claim 15, in which two or more gas supply devices which allow gas supply into the suspension at different container heights are present.

19. Device according to claim 15, in which the separation device is a funnel-shaped component, overflow arrangement, skimming device, suction device and/or a pneumatic device.

20. Hollow sphere having a multiaxially and elastically deformable wall composed of collagen and at least one collagen derivative, wherein at least the cavity formed is filled with at least one gas.

21. Hollow sphere according to claim 20, in which the collagen contains one or more different collagen types.

22. Hollow sphere according to claim 20, in which fibrillated collagen is present.

23. Hollow sphere according to claim 20, in which gelatin is present as the collagen derivative.

24. Hollow sphere according to claim 20, in which at least the cavity contains a liquid, microorganisms, solids and/or cellular tissue.

25. Hollow sphere according to claim 20, having an outer diameter of 0.2 mm to 1.5 cm.

26. Hollow sphere according to claim 20, in which the wall has a layer thickness of 5 μm to ≤500 μm, preferably 10 μm to 200 μm.
